(19) United States
(12) Patent Application Publication
(10) Pub. No.: US 2009/0239834 A1
(43) Pub. Date: Sep. 24, 2009

(54) MCC-257 MODULATION OF NEUROGENESIS

(75) Inventors: Carrolee Barlow, Del Mar, CA (US); Todd A. Carter, San Diego, CA (US); Andrew Morse, San Diego, CA (US); Kai Treuner, San Diego, CA (US); Kym I. Lorrain, San Diego, CA (US)

Correspondence Address: DLA PIPER LLP (US), 4365 EXECUTIVE DRIVE, SUITE 1100, SAN DIEGO, CA, 92121-2133, US

(73) Assignee: BrainCells, Inc., San Diego, CA (US)

(21) Appl. No.: 12/409,421

(22) Filed: Mar. 23, 2009

Related U.S. Application Data
(60) Provisional application No. 61/038,570, filed on Mar. 21, 2008.

(51) Int. Cl. A61K 31/58

(52) U.S. Cl. 514/172

(57) ABSTRACT

The disclosure provides methods and a composition for treating diseases and conditions of the central and peripheral nervous system by stimulating or increasing neurogenesis by use of (5-acetamido-N-(5alpha-cholestan-3alpha-yl)-3,5-dideoxy-2-O-methyl-D-glycero-alpha-D-galacto-non-2-ulopyranosonamide(MCC-257). The disclosure also includes methods and a composition for stimulating or activating the formation of new nerve cells based on the application of MCC-257

### CROSS-REFERENCES TO RELATED APPLICATIONS

This application claims priority benefit to U.S. Application Ser. No. 61/038,570 filed Mar. 21, 2008, the disclosure of which is hereby incorporated by reference in its entirety for all purposes.

### FIELD OF THE DISCLOSURE

The disclosure provides methods and a composition for treating diseases and conditions of the central and peripheral nervous system by stimulating or increasing neurogenesis by use of 5-acetamido-N-(5alpha-cholestan-3alpha-yl)-3,5-dideoxy-2-O-methyl-D-glycero-alpha-D-galacto-non-2-ulopyranosonamide (MCC-257). The disclosure also includes methods and compositions for stimulating or activating the formation of new nerve cells based on the application of MCC-257.

### BACKGROUND OF THE DISCLOSURE

Neurogenesis is a vital process in the brains of animals and humans, whereby new nerve cells are continuously generated throughout the life span of the organism. The newly born cells are able to differentiate into functional cells of the central nervous system and integrate into existing neural circuits in the brain. The subgranular zone of the hippocampus is one of only two major areas of the adult brain capable of generating new neurons (Gage “Mammalian neural stem cells.” Science 2000 287(5457):1433-8; Warner-Schmidt and Duman “Hippocampal neurogenesis: opposing effects of stress and antidepressant treatment.” Hippocampus 2006; 16(3):239-49). In these regions, multipotent neural progenitor cells (NPCs) continue to divide and give rise to new functional neurons and glial cells (Gage “Mammalian neural stem cells.” Science 2000 287(5457):1433-8). Hippocampal neurogenesis is an extremely dynamic process that is regulated by stress, endocrine, and pharmacological factors (Warner-Schmidt and Duman “Hippocampal neurogenesis: opposing effects of stress and antidepressant treatment.” Hippocampus 2006; 16(3):239-49). As such, a variety of factors can stimulate adult hippocampal neurogenesis, e.g., adrenalectomy, voluntary exercise, enriched environment and hippocampus dependent learning (Yehuda et al., “Enhanced brain cell proliferation following early adrenalectomy in rats.” J Neorochem 1989 53(1):241-8; van Praag et al. “Running increases cell proliferation and neurogenesis in the adult mouse dentate gyrus.” Nat. Neurosci. 1999 2(3) 266-70; Gould “Serotonin and hippocampal neurogenesis.” Neuropsychopharcology 1999 21(2 suppl):46S-51S; Malberg et al. “Chronic antidepressant treatment increases neurogenesis in adult rat hippocampus.” J Neurosci 2000 20(24):9104-10; Brown et al. “Transient expression of doublecortin during adult neurogenesis.” J Comp Neurol 2003 467(1):1-10; Santarelli et al. “Requirement of hippocampal neurogenesis for the behavioral effects of antidepressants.” Science 2003 301(5634):805-9), whereas other factors, such as adrenal hormones, stress, age and drugs of abuse negatively influence neurogenesis (Cameron and Gould “Adult neurogenesis is regulated by adrenal steroids in the dentate gyrus.” Neurosci 1994 61(2):203-9; Kuhn and Dickinson-Anson “Neurogenesis in the dentate gyrus of the adult rat: age-related decrease of neural progenitor proliferation.” J Neurosci 1996 16(6):2027-33; McEwen “Stress and hippocampal plasticity.” Annu Rev Neurosci 1999 22:105-22; Eisch and Mandyam “Drug dependence and addition, II: Adult neurogenesis and drug abuse.” Am J Psychiatry 2004 161(3):426). Recently, researchers have found that neurogenesis may contribute to the therapeutic effects of drugs used to treat neurological diseases. Therefore, what is need in the art are new drugs that are useful for increasing neuorogenesis.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### BRIEF SUMMARY OF THE DISCLOSURE

The disclosure provides methods and a composition for the prevention and treatment of diseases, conditions and injuries of the central and peripheral nervous systems by stimulating or increasing neurogenesis. Aspects of the methods and the activities of the composition includes stimulating, increasing, or potentiating neurogenesis in cases of a disease, disorder, or condition of the central and/or peripheral nervous system. Embodiments of the disclosure include methods for treating neurodegenerative disorders, neurological trauma to the brain or central nervous system including recovery, depression, anxiety, psychosis, learning and memory disorders and ischemia of the central and/or peripheral nervous systems. In other embodiments, the disclosed methods and compositions are useful for improving cognitive outcomes, anxiety and mood disorders.

The disclosure also provides methods and compositions for modulating neurogenesis, such as by stimulating, increasing or potentiating neurogenesis. The neurogenesis may be at the level of a cell or tissue. The cell or tissue may be present in an animal subject or more preferably a human subject, or alternatively be in an in vitro or ex vivo setting. In some embodiments, neurogenesis is stimulated or increased in a neural cell or tissue, such as that of the central or peripheral nervous system of an animal or human subject. In cases of an animal or human subject, the methods may be practiced in connection with one or more disease, disorder, or condition of the nervous system as present in the animal or human subject.

Thus, the embodiments disclosed herein include methods for treating a subject suffering from a nervous system disorder, disease, or condition by administering to the subject a therapeutically effective amount of 5-acetamido-N-(5alpha-cholestan-3alpha-yl)-3,5-dideoxy-2-O-methyl-D-glycero-alpha-D-galacto-non-2-ulopyranosonamide (MCC-257), or a pharmaceutically acceptable hydrate or solvate thereof. The subject may be an animal or human.

The disclosure may be practiced based on use of MCC-257, or a pharmaceutically acceptable hydrate or solvate thereof, as a “direct” agent, in that it has direct activity via interaction with its receptor(s) in cells, or as an “indirect” agent in that MCC-257, or a pharmaceutically acceptable hydrate or solvate thereof, does not directly interact with a receptor. An indirect agent may act on a receptor indirectly, or via production, generation, stabilization, or retention of an intermediate agent which then directly interacts with the receptor.

In another aspect, the disclosure provides methods for lessening and/or reducing a decline or decrease of cognitive function in a animal or human subject due to a nervous system disorder, disease or condition. In some cases, the method may be applied to maintain and/or stabilize cognitive function in the subject. Thus cognitive impairment may be the result of chronic infection, toxic disorders, neurodegenerative disorders and combinations thereof. In some embodiments disclosed herein, the methods comprise administering MCC-257, or a pharmaceutically acceptable hydrate or solvate thereof, to a subject in an amount effective to reduce or lessen cognitive impairment.

In another aspect, the disclosure provides methods for treating a subject suffering from cognitive impairment due to a non-disease state comprising administering to the subject a therapeutically effective amount of MCC-257 or pharmaceutically acceptable hydrate or solvate thereof. Non-limiting examples of non-disease states include cognitive impairment due to aging, chemotherapy and radiation therapy.

In another aspect, the disclosure provides methods for treating a mental disorder with use of MCC-257. In some embodiments, the method may be used to moderate or alleviate the mental disorder in an animal or human subject. Non-limiting examples of a mental disorder include an affective disorder including anxiety and depression. In other embodiments, the method may be used to improve, maintain, or stabilize affective disorder in a subject.

In another aspect, the disclosed methods include identifying an animal or human subject suffering from one or more diseases, disorders, or conditions, or a symptom thereof, and administering to the subject MCC-257 or a pharmaceutically acceptable hydrate or solvate thereof. In some embodiments, the disclosed methods include identification of a subject as in need of an increase in neurogenesis; and administering a therapeutically effective amount of MCC-257. In other embodiments, the subject is a mammal, more preferably a human being.

In another aspect, the disclosure provides methods for stimulating or increasing neurogenesis in a cell or tissue. The cell or tissue is contacted with an effective amount of MCC-257 or a pharmaceutically acceptable hydrate or solvate thereof to stimulate or increase neurogenesis in said cell or tissue. This cell or tissue may be in an animal or human subject having a condition affecting normal neurogenesis whereby stimulating or increasing neurogenesis improves the condition. Thus the cell or tissue to be treated may exhibit the effects of insufficient amounts of, inadequate levels of, or aberrant neurogenesis. In some embodiments, the subject may be one that has a disease, condition or disorder which results in suppressed or decreased neurogenesis. These subjects would have symptoms and conditions associated with decreased neurogenesis and thus would benefit from a process of stimulating, increasing or potentiating neurogenesis. A non limiting example of such condition is the reduction in, or impairment of, cognition, such as that due to a chronic infection, a neurodegenerative disease, head injury or a toxic disorder.

In another aspect, MCC-257 may be administered to an animal or human subject exhibiting the effects of aberrant neurogenesis. In some embodiments, the aberrant neurogenesis may be attributed to epilepsy, or a condition associated with epilepsy as non-limiting examples. Increased neurogenesis would alleviate the aberrant neurogenic symptoms in the subject.

In an additional aspect, MCC-257 may be administered to an animal or human subject that will be subjected to an agent that decreases or inhibits neurogenesis. Non-limiting examples of an inhibitor of neurogenesis include opioid receptor agonists, such as morphine (mu receptor subtype agonist). Non-limiting examples include administering MCC-257 to a subject before, simultaneously with, or after the subject has be administered morphine or other opiate in connection with a surgical procedure. Other non-limiting embodiments of instances where a subject may be administered MCC-257 before, simultaneously with, or after a procedure would include radiation therapy or chemotherapy.

In an additional aspect, the cells undergoing neurogenesis may by neural stem cells (NSCs). These neural stem cells may differentiate along a neuronal lineage, a glial lineage or both. In an additional embodiment of the disclosure the neural stem cells and/or neurogenesis may be in the hippocampus of the subject.

In yet another aspect, the disclosure provides methods for modulating neurogenesis, such as by stimulating or increasing neurogenesis, in an animal or human subject by administering MCC-257. In some embodiments, the neurogenesis occurs in combination with the stimulation of angiogenesis which provides new cells with access to the circulatory system.

The details of additional embodiments are set forth in the accompanying drawings and the description below. Other features, objects, and advantages of the embodiments will be apparent from the drawings, the detailed description, and from the claims.

### BRIEF DESCRIPTION OF THE DRAWINGS

FIG. 1 is a dose-response curve showing the effect of MCC-257 on neuronal differentiation of human neural stem cells. Data is presented as the percentage of the neuronal positive control, with basal media values subtracted. EC₅₀ was observed at an MCC-257 concentration of 0.09 μM in test cells, compared to 4.7 μM for the positive control compound.

FIG. 2 shows the effect of chronic dosing of male F344 rats with MCC-257 on latency to eat in a novelty-suppressed feeding assay. The white bar represents the vehicle control (DI water; n=10), the black bar represents fluoxetine dosed at 12.5 mg/kg (n=11) and the grey bar represents MCC-257 dosed at 0.3 mg/kg (n=11). Vehicle and compounds were administered orally once daily (q.d.) for 20 days prior to behavioral testing. Behavioral testing was carried out as described in Example 2. Results indicate the mean latency (seconds) to approach and eat a food pellet within a novel environment. Fluoxetine and the 0.3 mg/kg dose of MCC-257 significantly reduced latency to eat relative to vehicle controls (p<0.05; one-tailed Student's t-test). Values are the mean±SEM.

FIG. 3 shows the effect of chronic dosing of rats with MCC-257 on the differentiation of neural progenitor cells into mature neurons within the dentate gyrus of the hippocampus. The white bar represents vehicle (DI water, n=11) and the grey bar represents MCC-257 dosed at 0.3 mg/kg (n=10). Both vehicle and MCC-257 were dosed orally once daily (q.d.) for 26 days prior to analysis. The results show BrdU positive cell counts within the granule cell layer of the dentate gyrus. Data are presented as the number of BrdU cells per cubic mm of dentate gyrus. MCC-257 dosed at 0.3 mg/kg significantly increased neurogenesis relative to the vehicle controls (p=0.03; one-tailed Student's t-test). Values are the mean±SEM.

FIG. 4 shows the effect of chronic dosing of rats with MCC-257 on their behavior in the Novel Object Recognition (NOR) cognition assay following a 48-hour delay. Behavioral testing was carried out as described in Example 2. Data is presented as the mean percentage of visits to a novel object. The white bar represents vehicle (DI water: n=11) and grey bars represent increasing doses of MCC-257 (0.1 mg/kg, n=10; 0.3 mg/kg, n=10; 3.0 mg/kg, n=10). All animals were dosed orally once daily (q.d.) for 29 days prior to testing. Treatment with MCC-257 resulted in dose-related increase in preference for the novel object (indicative of cognitive enhancement). Values are the mean±SEM.

### DETAILED DESCRIPTION OF THE DISCLOSURE

“MCC-257” (5-acetamido-N-(5alpha-cholestan-3alpha-yl)-3,5-dideoxy-2-O-methyl-D-glycero-alpha-D-galacto-non-2-ulopyranosonamide) refers a neuroprotective sialic acid derivative developed by the Mitsubishi Tanabe Pharma Corporation having the following structure.

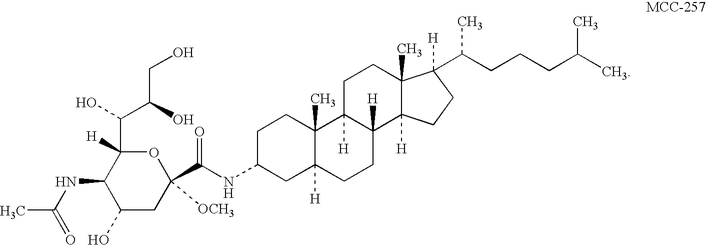

As used herein “MCC-257” refers to the unaltered compound as well as a pharmaceutically acceptable hydrate or solvate thereof. MCC-257 is described in U.S. Pat. No. 5,712,254.

“Neurogenesis” is defined herein as proliferation, differentiation, migration and/or survival of a neural cell in vivo or in vitro. In some embodiments, the neural cell is an adult, fetal, or embryonic neural stem cell or population of cells. The cells may be located in the central nervous system or elsewhere in an animal or human being. The cells may also be in a tissue, such as neural tissue. In some embodiments, the neural cell is an adult, fetal, or embryonic progenitor cell or population of cells, or a population of cells comprising a mixture of stem cells and progenitor cells. Neural cells include all brain stem cells, all brain progenitor cells, and all brain precursor cells. Neurogenesis includes neurogenesis as it occurs during normal development, as well as neural regeneration that occurs following disease, damage or therapeutic intervention, such as by the treatment described herein.

The term “astrogenic” is defined in relation to “astrogenesis” which refers to the activation, proliferation, differentiation, migration and/or survival of an astrocytic cell in vivo or in vitro. Non-limiting examples of astrocytic cells include astrocytes, activated microglial cells, astrocyte precursors and potentiated cells, and astrocyte progenitor and derived cells. In some embodiments, the astrocyte is an adult, fetal, or embryonic astrocyte or population of astrocytes. The astrocytes may be located in the central nervous system or elsewhere in an animal or human being. The astrocytes may also be in a tissue, such as neural tissue. In some embodiments, the astrocyte is an adult, fetal, or embryonic progenitor cell or population of cells, or a population of cells comprising a mixture of stem and/or progenitor cells, which is/are capable of developing into astrocytes. Astrogenesis includes the proliferation and/or differentiation of astrocytes as it occurs during normal development, as well as astrogenesis that occur following disease, damage or therapeutic intervention.

The term “stem cell” (or neural stem cell (NSC)), as used herein, refers to an undifferentiated cell that is capable of self-renewal and differentiation into neurons, astrocytes, and/or oligodendrocytes.

The term “progenitor cell” (e.g., neural progenitor cell), as used herein, refers to a cell derived from a stem cell that is not itself a stem cell. Some progenitor cells can produce progeny that are capable of differentiating into more than one cell type.

The terms “animal subject” refers to a non-human mammals, such as a primate, canine, or feline. In other embodiments, the terms refer to an animal that is domesticated (e.g. livestock) or otherwise subject to human care and/or maintenance (e.g. zoo animals and other animals for exhibition). In other non-limiting examples, the terms refer to ruminants or carnivores, such as dogs, cats, birds, horses, cattle, sheep, goats, marine animals and mammals, penguins, deer, elk, and foxes.

The term “condition” refers to the physical and/or psychological state of an animal or human subject selected for treatment with the disclosed compound or compounds. The physical and/or psychological state of the animal or human subject at the time of treatment may include but is not limited to a disease state, a disease symptom, and/or a disease syndrome. The physical and/or psychological state of the animal or human subject may be the result of an injury, disease or disorder and/or a result of treating such injury, disease or disorder.

The term “nervous system disorder” refers to diseases and disorders of the nervous system categorized under “mental disorders” or “diseases and disorders of the central nervous system”.

The term “mental disorder” refers to a group of disorders that are commonly associated with an anxiety disorder, a mood disorder or schizophrenia as disclosed in “Harrison's Principles of Internal Medicine” 17^{th} edition, which is herein incorporated in its entirety.

The term “affective disorder” as used herein encompasses depression and anxiety. An “affective disorder” comprises the symptoms of depression and/or anxiety. The novelty suppressed feeding assay as used herein is a model used for identifying anxiolytics and antidepressants.

The term “anxiety disorder” refers to or connotes significant distress and dysfunction due to feelings of apprehension, guilt, fear, and the like. Anxiety disorders include, but are not limited to panic disorders, posttraumatic stress disorder, obsessive-compulsive disorder and phobic disorders.

The term “mood disorder” is typically characterized by pervasive, prolonged, and disabling exaggerations of mood, which are associated with behavioral, physiologic, cognitive, neurochemical and psychomotor dysfunctions. As used herein a mood disorder includes but is not limited to bipolar disorders, depression including major depressive disorder, and depression associated with various disease states and injuries.

The term “diseases and disorders of the central nervous system” include but are not limited to epilepsy, cerebrovascular disease, cognitive impairment, neuropathy, myelopathy and head injury as disclosed in “Harrison's Principles of Internal Medicine” 17^{th} edition, which is incorporated in its entirety.

As used herein, the term “neurodegenerative disorder” encompasses diseases and disorders of the central nervous system wherein neuronal perturbations are the result of the disease or disorder. As non-limiting examples of neuronal perturbations are those noted within the hippocampus resulting in decreased neurogenesis, aberrant neurogenesis, as well as defects to neuronal and synaptic plasticity.

As used herein, the term “cognitive impairment” refers to diminished or reduced cognitive function. This may be the result of a number of natural and physical events including but not limited to aging, head trauma, diseases and disorders of the central nervous system, therapies related to treating a disease or disorder (drugs, chemotherapy and radiation therapy), as well as alcohol and drug abuse.

The term “cognitive function” refers to high-level brain functions of an animal or human subject relating to information gathering and/or processing; the understanding, reasoning, and/or application of information and/or ideas; the abstraction or specification of ideas and/or information; acts of creativity, problem-solving, and possibly intuition; and mental processes such as learning, perception, and/or awareness of ideas and/or information. The mental processes are distinct from those of beliefs, desires, and the like. In some embodiments, cognitive function may be assessed, and thus optionally defined, via one or more tests or assays for cognitive function. Non-limiting examples of a test or assay for cognitive function include CANTAB (see for example Fray et al. “CANTAB battery: proposed utility in neurotoxicology.” Neurotoxicol Teratol. 1996; 18(4):499-504), Stroop Test, Trail Making, Wechsler Digit Span, or the CogState computerized cognitive test (see also Dehaene et al. “Reward-dependent learning in neuronal networks for planning and decision making.” Prog Brain Res. 2000; 126:217-29; Iverson et al. “Interpreting change on the WAIS-III/WMS-III in clinical samples.” Arch Clin Neuropsychol. 2001; 16(2):183-91; and Weaver et al. “Mild memory impairment in healthy older adults is distinct from normal aging.” Brain Cogn. 2006; 60(2):146-55). The novel object recognition assay as used herein is a model used for screening potential compounds having an effect on cognitive function.

The term “dementia” is the progressive decline in cognitive function due to damage or disease in the body beyond what might be expected from normal aging. Dementia is a non-specific illness syndrome in which affected areas of cognition may be memory, attention, language, and problem solving.

As used herein, “treating” includes prevention, amelioration, alleviation, and/or elimination of the disease, disorder, or condition being treated or one or more symptoms of the disease, disorder, or condition being treated, as well as improvement in the overall well being of a patient, as measured by objective and/or subjective criteria. In some embodiments, treating is used for reversing, attenuating, minimizing, suppressing, or halting undesirable or deleterious effects of, or effects from the progression of, a disease, disorder, or condition of the central and/or peripheral nervous systems. In other embodiments, the method of treating may be advantageously used in cases where additional neurogenesis would replace, replenish, or increase the numbers of cells lost due to injury or disease as non-limiting examples.

“IC₅₀” and “EC₅₀” values are concentrations of MCC-257, which reduce and promote, respectively, neurogenesis or another physiological activity (e.g., the activity of a receptor) to a half-maximal level. IC₅₀ and EC₅₀ values may be assayed in a variety of environments, including cell-free environments, cellular environments (e.g., cell culture assays), multicellular environments (e.g., in tissues or other multicellular structures), and/or in vivo. In some embodiments, MCC-257 has IC₅₀ or EC₅₀ values of less than about 10 μM, less than about 1 μM, or less than about 0.1 μM or lower. In other embodiments, MCC-257, has an IC₅₀ of less than about 50 nM, less than about 10 nM, or less than about 1 nM or lower.

In some embodiments, the selectivity of MCC-257, is individually measured as the ratio of the IC₅₀ or EC₅₀ value for a desired effect (e.g., modulation of neurogenesis) relative to the IC₅₀/EC₅₀ value for an undesired effect.

In some cases, administration of MCC-257, results in improved efficacy, fewer side effects, lower effective dosages, less frequent dosing, and/or other desirable effects.

In other embodiments, modulation by MCC-257, is in a region containing neural cells affected by disease or injury, a region containing neural cells associated with disease effects or processes, or a region containing neural cells affected by another event injurious to neural cells. Non-limiting examples of such events include stroke, radiation therapy and chemotherapy.

The methods described herein may be used to treat any disease or condition for which it is beneficial to promote or otherwise stimulate or increase neurogenesis. One focus of the methods described herein is to achieve a therapeutic result by stimulating, increasing or potentiating neurogenesis via use of MCC-257. Thus, certain methods described herein may be used to treat any disease or condition susceptible to treatment by increasing neurogenesis.

Within the scope of the disclosure are methods applied to modulating neurogenesis in vivo, in vitro, or ex vivo. In in vivo embodiments, the cells may be present in a tissue or organ of a subject animal or human being. Non-limiting examples of cells include those capable of neurogenesis, such as to result, whether by differentiation or by a combination of differentiation and proliferation, in differentiated neural cells. As described herein, neurogenesis includes the differentiation of neural cells along different potential lineages. In some embodiments, the differentiation of neural stem or progenitor cells is along a neuronal cell lineage to produce neurons. In other embodiments, the differentiation is along both neuronal and glial cell lineages.

In additional embodiments, the disclosure further includes differentiation along a neuronal cell lineage to the exclusion of one or more cell types in a glial cell lineage. Non-limiting examples of glial cell types include oligodendrocytes and radial glial cells, as well as astrocytes, which have been reported as being of an “astroglial lineage.” Therefore, embodiments of the disclosure include differentiation along a neuronal cell lineage to the exclusion of one or more cell types selected from oligodendrocytes, radial glial cells, and astrocytes.

In further embodiments, the methods described herein may allow for treatment of diseases characterized by pain, addiction, and/or depression by directly replenishing, replacing, and/or supplementing neurons and/or glial cells. In further embodiments, the methods described herein may enhance the growth and/or survival of existing neural cells, and/or slow or reverse the loss of such cells in a neurodegenerative condition.

Where a method comprises contacting a neural cell with MCC-257, the result may be an increase in neuro-differentiation. The method may be used to potentiate a neural cell for proliferation, and thus neurogenesis, via administration of MCC-257. Thus, the disclosure includes methods for maintaining, stabilizing, stimulating increasing or potentiating neurodifferentiation in a cell or tissue by use of MCC-257. The method may comprise contacting a cell or tissue with MCC-257, to maintain, stabilize stimulate, increase or potentiate neurodifferentiation in the cell or tissue.

In another aspect, the disclosure provides methods for stimulating or increasing neurogenesis in a cell or tissue, by contacting the cell or tissue with MCC-257, wherein the effect is to produce neurogenesis in the cell or tissue, and wherein the neurogenesis comprises differentiation of neural stem cells (NSCs) along a neuronal lineage or along a glial cell line.

The disclosure also includes methods comprising contacting the cell or tissue with MCC-257, which stimulates or increases proliferation or cell division in a neural cell. The increase in neuroproliferation may be due to the MCC-257. In some cases, the disclosed methods comprising MCC-257, may be used to produce neurogenesis (in this case both neurodifferentiation and/or proliferation) in a population of neural cells. In some embodiments, the cell or tissue is in an animal subject or a human patient. Non-limiting examples of conditions in need of neurogenesis include a human patient treated with chemotherapy and/or radiation, or other therapy or condition which is detrimental to cognitive function; or a human patient diagnosed with a degenerative disease; or a human patient diagnosed as having epilepsy, a condition associated with epilepsy, or seizures associated with epilepsy.

In another aspect, the disclosure provides methods for stimulating or increasing neurogenesis in a cell or tissue, by contacting the cell or tissue with MCC-257, wherein the effect is to produce neurogenesis in the cell or tissue, wherein the cell or tissue is in an animal subject or a human patient, and wherein the subject or patient has one or more chemical addiction or dependency.

In another aspect, the disclosure provides methods for treating a nervous system disorder related to a mental disorder or a disease or disorder of the central nervous system in a subject or patient, the method comprising administering MCC-257 to the subject or patient, wherein the effect is to produce an improvement in the disorder or disease in the subject or patient.

In another aspect, the disease or disorder of the central nervous system to be treated is selected from epilepsy, cerebrovascular ischemia, cognitive impairment, neuropathy, myelopathy head injury or other neurologically related disorder.

In another aspect, the disclosed methods may be used to moderate or alleviate a mental disorder in a subject or patient. The mental disorder to be treated may be selected from an affective disorder or schizophrenia. As used herein an affective disorder encompasses anxiety and depression. Thus, the disclosure includes methods for treating a mental disorder in a subject or patient by administering a therapeutically effective amount of MCC-257. Non-limiting examples of the method include the administration of MCC-257 to a subject of patient that is under a treatment and/or has a condition that results in a mental disorder.

In some embodiments, wherein the subject or patient demonstrates impaired cognitive function, methods of the disclosure may be useful for enhancing or improving the cognitive impairment. The methods may comprise administering MCC-257 to a subject or patient to enhance or improve a decline or decrease of cognitive function due to a therapy and/or condition that reduces cognitive function. Non-limiting examples and conditions affecting cognitive impairment are aging, chronic infections, toxic disorders, degenerative disorders or combinations thereof. Cognitive impairment due to age may be age-associated memory impairment (AAMI), age-associated cognitive decline (AACD), mild cognitive impairment or age-related memory loss. Non-limiting examples of chronic infections affecting cognition are HIV or Creutzfeldt-Jakob disease. Non-limiting examples of toxic disorders affecting cognition are radiation therapy, chemotherapy, drug or alcohol abuse and combinations thereof. Non-limiting examples of degenerative disorders affecting cognition are Alzheimer's disease, Huntington's disease, Parkinson's disease, Multiple Sclerosis and combinations thereof.

Other methods of the disclosure include treatment to affect or maintain the cognitive function of a subject or patient. In some embodiments, the maintenance or stabilization of cognitive function may be at a level, or thereabouts, present in a subject or patient in the absence of a therapy and/or condition that reduces cognitive function. In alternative embodiments, the maintenance or stabilization may be at a level, or thereabouts, present in a subject or patient as a result of a therapy and/or condition that reduces cognitive function.

The disclosed methods may optionally include assessing or measuring cognitive function of the subject or patient before, during, and/or after administration of the treatment to detect or determine the effect thereof on cognitive function. In one embodiment, the disclosed methods may comprise i) treating a subject or patient that has been previously assessed for cognitive function and ii) reassessing cognitive function in the subject or patient during or after the course of treatment. The assessment may measure cognitive function for comparison to a control or standard value (or range) in subjects or patients in the absence of MCC-257. This may be used to assess the efficacy of MCC-257 in alleviating the reduction in cognitive function.

In another aspect, the disclosure provides methods for stimulating or increasing neurogenesis in a cell or tissue, by contacting the cell or tissue with MCC-257, wherein the effect is to produce neurogenesis in the cell or tissue, wherein MCC-257 is in a pharmaceutically acceptable formulation.

The disclosure includes methods for the identification of an individual suffering from one or more disease, disorder, or condition, or a symptom thereof, and administering to the subject or patient a therapeutically effective amount of MCC-257. The identification of a subject or patient as having one or more disease, disorder or condition, or a symptom thereof, may be made by a skilled practitioner using any appropriate means known in the field.

In some embodiments, the identification of a patient in need of neurogenic modulation comprises identifying a patient who has or will be exposed to a factor or condition known to inhibit neurogenesis, including but not limited to, stress, aging, sleep deprivation, hormonal changes (e.g., those associated with puberty, pregnancy), or aging (e.g., menopause), lack of exercise, lack of environmental stimuli (e.g., social isolation), diabetes and drugs of abuse (e.g., alcohol, especially chronic use; opiates and opioids; psychostimulants). In some cases, the patient has been identified as non-responsive to treatment with primary medications for the condition(s) targeted for treatment (e.g., non-responsive to antidepressants for the treatment of depression), and MCC-257 may be administered in a method for enhancing the responsiveness of the patient to a co-existing or pre-existing treatment regimen.

In additional embodiments, the patient in need of neurogenic modulation suffers from premenstrual syndrome, post-partum depression, or pregnancy-related fatigue and/or depression, and the treatment comprises administering a therapeutically effective amount of MCC-257. Without being bound by any particular theory, and offered to improve understanding of the disclosure, it is believed that levels of steroid hormones, such as estrogen, are increased during the menstrual cycle during and following pregnancy, and that such hormones can exert a modulatory effect on neurogenesis.

In some embodiments, the patient is a user of a recreational drug including but not limited to alcohol, amphetamines, PCP, cocaine, and opiates. Without being bound by any particular theory, and offered to improve understanding of the disclosure, it is believed that some drugs of abuse have a modulatory effect on neurogenesis, which is associated with depression, anxiety and other mood and affective disorders, as well as deficits in cognition, learning, and memory. Moreover, mood disorders are causative/risk factors for substance abuse, and substance abuse is a common behavioral symptom (e.g., self medicating) of mood disorders. Thus, substance abuse and mood disorders may reinforce each other, rendering patients suffering from both conditions non-responsive to treatment. Thus, in some embodiments, MCC-257 may be administered to treat patients suffering from substance abuse and/or mood disorders.

In further embodiments, the patient is on a co-existing and/or pre-existing treatment regimen involving administration of one or more prescription medications having a modulatory effect on neurogenesis. For example, the patient suffers from chronic pain and is prescribed one or more opiate/opioid medications; and/or suffers from ADD, ADHD, or a related disorder, and is prescribed a psychostimulant, such as ritalin, dexedrine, adderall, or a similar medication. Without being bound by any particular theory, and offered to improve understanding of the disclosure, it is believed that such medications may exert a modulatory effect on neurogenesis, leading to depression, anxiety and other mood disorders, as well as deficits in cognition, learning, and memory. Thus, MCC-257 may be administered to patients receiving these prescribed medications in order to treat the associated depression, anxiety, and/or other mood disorders, and/or to improve cognition.

In additional embodiments, the patient suffers from chronic fatigue syndrome; a sleep disorder; lack of exercise (e.g., elderly, infirm, or physically handicapped patients); and/or lack of environmental stimuli (e.g., social isolation); and the treatment comprises administering a therapeutically effective amount of MCC-257.

In more embodiments, the patient is an individual having, or who is likely to develop, a disorder related to neural degeneration, neural damage and/or neural demyelination.

In yet additional embodiments, identifying a patient in need of neurogenesis modulation comprises selecting a population or sub-population of patients, or an individual patient, that is more amenable to treatment and/or less susceptible to side effects than other patients having the same disease or condition. In some embodiments, identifying a patient amenable to treatment with MCC-257, comprises identifying a patient who has been exposed to a factor known to enhance neurogenesis, including but not limited to, exercise, hormones or other endogenous factors, and drugs taken as part of a pre-existing treatment regimen.

In some embodiments, a sub-population of patients is identified as being more amenable to neurogenesis modulation with MCC-257, by taking a cell or tissue sample from prospective patients, isolating and culturing neural cells from the sample, and determining the effect of MCC-257 on the degree or nature of neurogenesis of the cells, thereby allowing selection of patients for which the therapeutic agent has a substantial effect on neurogenesis. Advantageously, the selection of a patient or population of patients in need of or amenable to treatment with MCC-257, allows for more effective treatment of the disease or condition targeted for treatment than known methods using the same or similar compounds.

In some embodiments, the patient has suffered a CNS insult, such as a CNS lesion, a seizure (e.g., electroconvulsive seizure treatment; epileptic seizures), radiation therapy, chemotherapy and/or stroke or other ischemic injury. Without being bound by any particular theory, and offered to improve understanding of the disclosure, it is believed that some CNS insults/injuries leads to increased proliferation of neural stem cells, but that the resulting neural cells form aberrant connections which can lead to impaired CNS function and/or diseases, such as temporal lobe epilepsy. In other embodiments, MCC-257 may be administered to a patient who has suffered, or is at risk of suffering, a CNS insult or injury to stimulate neurogenesis.

Advantageously, stimulation of the differentiation of neural stem cells with MCC-257, activates signaling pathways necessary for progenitor cells to effectively migrate and incorporate into existing neural networks or to block inappropriate proliferation.

Additionally, the disclosed methods provide for the application of MCC-257 to treat a subject or patient for a condition due to the anti-neurogenic effects of an opiate or opioid based analgesic.

In some embodiments, the administration of an opiate or opioid based analgesic, such as an opiate like morphine or other opioid receptor agonist, to a subject or patient results in a decrease in, or inhibition of, neurogenesis. The administration of MCC-257 would reduce the anti-neurogenic effect.

The disclosed embodiments include a method of treating post operative pain in a subject or patient by combining the administration of MCC-257.

Other disclosed embodiments include methods for treating or preventing decreases in, or inhibition of, neurogenesis resulting from the use of an opioid receptor agonist. The methods comprise the administration of a therapeutically effective amount of MCC-257. Non-limiting examples include cases involving an opioid receptor agonist, which decreases or inhibits neurogenesis, and drug addiction, drug rehabilitation, and/or prevention of relapse into addiction. In some embodiments, the opioid receptor agonist is morphine, opium or other opiate.

In additional embodiments, the disclosed methods may be used to treat subjects having, or diagnosed with, depression or other withdrawal symptoms from morphine or other agents which decrease or inhibit neurogenesis. This is distinct from the treatment of subjects having, or diagnosed with, depression independent of an opiate, such as that of a mental disorder, as disclosed herein. In further embodiments, the methods may be used to treat a subject with one or more chemical addiction or dependency, such as with morphine or other opiates, where the addiction or dependency is ameliorated or alleviated by an increase in neurogenesis.

In other embodiments, the methods described herein involve modulating neurogenesis in vitro or ex vivo with MCC-257, such that a composition containing neural stem cells, neural progenitor cells, and/or differentiated neural cells can subsequently be administered to an individual to treat a disease or condition.

In some embodiments, the method of treatment comprises the steps of contacting a neural stem cell or progenitor cell with MCC-257, to modulate neurogenesis, and transplanting the cells into a patient in need of treatment. Methods for transplanting stem and progenitor cells are known in the art, and are described, e.g., in U.S. Pat. Nos. 5,928,947; 5,817,773; and 5,800,539, and PCT Publication Nos. WO 01/176507 and WO 01/170243, all of which are incorporated herein by reference in their entirety. In some embodiments, methods described herein allow treatment of diseases or conditions by directly replenishing, replacing, and/or supplementing damaged or dysfunctional neurons. In further embodiments, methods described herein enhance the growth and/or survival of existing neural cells, and/or slow or reverse the loss of such cells in a neurodegenerative or other condition.

In alternative embodiments, the method of treatment comprises identifying, generating, and/or propagating neural cells in vitro or ex vivo in contact with MCC-257, and transplanting the cells into a subject.

In another embodiment, the method of treatment comprises the steps of contacting a neural stem cell of progenitor cell with MCC-257, to stimulate neurogenesis or neurodifferentiation, and transplanting the cells into a patient in need of treatment. Also disclosed are methods for preparing a population of neural stem cells suitable for transplantation, comprising culturing a population of neural stem cells (NSCs) in vitro, and contacting the cultured neural stem cells with MCC-257. The disclosure further includes methods of treating the diseases, disorders, and conditions described herein by transplanting such treated cells into a subject or patient.

In additional embodiments, the disclosure includes a method of stimulating or increasing neurogenesis in a subject or patient with stimulation of angiogenesis in the subject or patient. The co-stimulation may be used to provide the differentiating and/or proliferating cells with increased access to the circulatory system. The neurogenesis is produced by administering MCC-257.

As described herein, the disclosed embodiments include methods for treating diseases, disorders, and conditions of the central and/or peripheral nervous systems (CNS and PNS, respectively) by administering MCC-257. The amount of MCC-257 may be any that results in a measurable relief of a disease condition like those described herein. As a non-limiting example, an improvement in the Hamilton depression scale (HAM-D) score for depression may be used to determine (such as quantitatively) or detect (such as qualitatively) a measurable level of improvement in the depression of a subject.

Non-limiting examples of symptoms that may be treated with the methods described herein include abnormal behavior, abnormal movement, hyperactivity, hallucinations, acute delusions, combativeness, hostility, negativism, withdrawal, seclusion, memory defects, sensory defects, cognitive defects, and tension. Non-limiting examples of abnormal behavior include irritability, poor impulse control, distractibility, and aggressiveness. Outcomes from treatment with the disclosed methods include improvements in cognitive function or capability in comparison to the absence of treatment.

Additional examples of diseases, disorders, and conditions of the central and/or peripheral nervous systems (CNS and PNS, respectively) and associated conditions treatable by the methods described herein include, but are not limited to, neurodegenerative diseases and disorders. Such diseases and disorders include but are not limited to Alzheimer's disease, Parkinson's disease, Huntington's disease (Huntington's Chorea), Lou Gehrig's disease, Pick's disease, epilepsy (seizures), multiple sclerosis, amyotrophic lateral sclerosis, progressive subcortical gliosis, progressive supranuclear palsy, thalmic degeneration syndrome, hereditary aphasia, Shy-Drager syndrome, Lewy body disease, cardiovascular diseases and conditions (i.e. infarcts, hemorrhage, cardiac disorders), mixed vascular, bacterial meningitis, Creutzfeld-Jacob Disease, and Cushing's disease, head injury, HIV disease and the conditions associated with such diseases and disorders such as but not limited to cognition, dementias (i.e. Parkinsonism dementia syndrome, senile dementia, memory disturbances/memory loss), delirium, amnestic disorders, depression and anxiety. In practice, a subject or patient may be afflicted with, or diagnosed with, one or more of the above mentioned diseases or disorders in any combination.

The disclosed embodiments also provide for the treatment of a nervous system disorder related to neural damage, cellular degeneration, mental disorders, cellular or tissue (neurological) trauma and/or injury (e.g., subdural hematoma or traumatic brain injury), toxic chemicals (e.g., heavy metals, alcohol, some medications), CNS hypoxia, or other neurologically related conditions. In practice, a therapeutically effective amount of MCC-257 may be administered to a subject or patient afflicted with, or diagnosed with, one or more central or peripheral nervous system disorders. Diagnosis may be performed by a skilled person in the applicable fields using known and routine methodologies which identify and/or distinguish these nervous system disorders from other conditions.

Non-limiting examples of nervous system disorders related to cellular degeneration include neurodegenerative disorders, neural stem cell disorders, neural progenitor cell disorders, and ischemic disorders. In some embodiments, an ischemic disorder comprises an insufficiency, or lack, of oxygen or angiogenesis, and non-limiting example include spinal ischemia, ischemic stroke, cerebral infarction, multi-infarct dementia. While these conditions may be present individually in a subject or patient, the disclosed methods also provide for the treatment of a subject or patient afflicted with, or diagnosed with, more than one of these conditions.

Non-limiting embodiments of nervous system disorders related to a mental disorder include affective disorders and schizophrenia. As used herein, an affective disorder refers to but is not limited to anxiety and depression. In practice, a subject or patient may be afflicted with, or diagnosed with, one or more of the above mentioned mental disorders in any combination.

Examples of nervous system disorders related to cellular or tissue trauma and/or injury include, but are not limited to neurological traumas and injuries, surgery related trauma and/or injury, retinal injury and trauma, injury related to epilepsy, spinal cord injury, brain injury, brain surgery, trauma related brain injury, trauma related to spinal cord injury, brain injury related to chemotherapy, spinal cord injury related to chemotherapy, brain injury related to radiation therapy, spinal cord injury related to radiation therapy, brain injury related to infection, spinal cord injury related to infection, brain injury related to inflammation, spinal cord injury related to inflammation, brain injury related to environmental toxin, and spinal cord injury related to environmental toxin and the conditions associated with such cellular or tissue trauma and/or injuries such as but not limited to cognition, dementias (i.e. dementia, memory disturbances and memory loss), delirium, amnestic disorders, mood disorders and anxiety disorders. In practice, a subject or patient may be afflicted with, or diagnosed with, one or more of the above mentioned cellular or tissue traumas and/or injuries in any combination.

Non-limiting examples of nervous system disorders related to other neurologically related conditions include learning disorders, memory disorders, age-associated memory impairment (AAMI) or age-related memory loss, autism, learning or attention deficit disorders (ADD or attention deficit hyperactivity disorder, ADHD), narcolepsy, sleep disorders and sleep deprivation (e.g., insomnia, chronic fatigue syndrome), cognitive disorders, epilepsy, injury related to epilepsy, and temporal lobe epilepsy and combinations thereof.

Other non-limiting examples of diseases and conditions treatable by the methods described herein include, but are not limited to, hormonal changes (e.g., depression and other mood disorders associated with puberty, pregnancy, or aging (e.g., menopause)); and lack of exercise (e.g., depression or other mental disorders in elderly, paralyzed, or physically handicapped patients); infections (e.g., HIV); genetic abnormalities (down syndrome); metabolic abnormalities (e.g., vitamin B12 or folate deficiency); hydrocephalus; memory loss separate from dementia, including mild cognitive impairment (MCI), age-related cognitive decline, and memory loss resulting from the use of general anesthetics, chemotherapy, radiation treatment, post-surgical trauma, or therapeutic intervention; and diseases of the of the peripheral nervous system (PNS), including but not limited to, PNS neuropathies (e.g., vascular neuropathies, diabetic neuropathies, amyloid neuropathies, and the like), neuralgias, neoplasms, myelin-related diseases, and the like.

Other conditions that may be beneficially treated by increasing neurogenesis are known in the art (see e.g., U.S. Publication Nos. 20020106731, 2005/0009742 and 2005/0009847, 20050032702, 2005/0031538, 2005/0004046, 2004/0254152, 2004/0229291, and 2004/0185429, herein incorporated by reference in their entirety).

MCC-257 may include a formulation such as a controlled release or timed release formulation. In other embodiments, the formulation may be for delivery via mode of administration and/or via tissue type. One non-limiting example is a chewing gum formulation containing MCC-257.

MCC-257 includes pharmaceutically acceptable hydrates, solvates and metabolites of this agent. Methods for preparing and administering hydrates, solvates, and metabolites of various agents are well known in the art.

Methods for assessing the nature and/or degree of neurogenesis in vivo and in vitro, for detecting changes in the nature and/or degree of neurogenesis, for identifying neurogenesis modulating agents, for isolating and culturing neural stem cells, and for preparing neural stem cells for transplantation or other purposes are disclosed, for example, in U.S. Provisional Application No. 60/697,905, and U.S. Publication Nos. 2005/0009742 and 2005/0009847, 20050032702, 2005/0031538, 2005/0004046, 2004/0254152, 2004/0229291, and 2004/0185429, all of which are herein incorporated by reference in their entirety.

In some embodiments of the disclosure, MCC-257 is in the form of a composition that includes at least one pharmaceutically acceptable excipient. As used herein, the term “pharmaceutically acceptable excipient” includes any excipient known in the field as suitable for pharmaceutical application. Suitable pharmaceutical excipients and formulations are known in the art and are described, for example, in Remington's Pharmaceutical Sciences (19th ed.) (Genarro, ed. (1995) Mack Publishing Co., Easton, Pa.). Pharmaceutical carriers may be chosen based upon the intended mode of administration of MCC-257. The pharmaceutically acceptable carrier may include, for example, disintegrants, binders, lubricants, glidants, emollients, humectants, thickeners, silicones, flavoring agents, and water.

MCC-257 may be incorporated with excipients and administered in the form of ingestible tablets, buccal tablets, troches, capsules, elixirs, suspensions, syrups, wafers, or any other form known in the pharmaceutical arts. The pharmaceutical compositions may also be formulated in a sustained release form. Sustained release compositions, enteric coatings, and the like are known in the art. Alternatively, the compositions may be a quick release formulation.

The amount of MCC-257 may be an amount that also potentiates or sensitizes, such as by activating or inducing cells to differentiate, a population of neural cells for neurogenesis. The degree of potentiation or sensitization for neurogenesis may be determined with use of MCC-257 in any appropriate neurogenesis assay, including, but not limited to, a neuronal differentiation assay described herein. In some embodiments, the amount of MCC-257 is based on the highest amount of MCC-257 that produces no detectable neuroproliferation in vitro but yet produces neurogenesis, or a measurable shift in efficacy in promoting neurogenesis in vitro.

As disclosed herein, an effective amount of MCC-257 in the described methods is an amount sufficient, when used as described herein, to stimulate or increase neurogenesis in the subject targeted for treatment when compared to the absence of MCC-257. An effective amount of MCC-257 may vary based on a variety of factors, including but not limited to, the physiological characteristics of the subject, the nature of the condition to be treated, and the route and/or method of administration. General dosage ranges of MCC-257 are provided herein and in the cited references based on animal models of CNS diseases and conditions. Various conversion factors, formulas, and methods for determining human dose equivalents of animal dosages are known in the art, and are described, e.g., in Freireich et al., Cancer Chemother Repts 50(4): 219 (1966), Monro et al., Toxicology Pathology, 23: 187-98 (1995), Boxenbaum and Dilea, J. Clin. Pharmacol. 35: 957-966 (1995), and Voisin et al., Reg. Toxicol. Pharmacol., 12(2): 107-116 (1990), which are herein incorporated by reference.

The disclosed methods typically involve the administration of MCC-257 in a dosage range of from about 0.001 ng/kg/day to about 200 mg/kg/day. Other non-limiting dosages include from about 0.001 to about 0.01 ng/kg/day, about 0.01 to about 0.1 ng/kg/day, about 0.1 to about 1 ng/kg/day, about 1 to about 10 ng/kg/day, about 10 to about 100 ng/kg/day, about 100 ng/kg/day to about 1 μg/kg/day, about 1 to about 2 μg/kg/day, about 2 μg/kg/day to about 0.02 mg/kg/day, about 0.02 to about 0.2 mg/kg/day, about 0.2 to about 2 mg/kg/day, about 2 to about 20 mg/kg/day, or about 20 to about 200 mg/kg/day. However, as understood by those skilled in the art, the exact dosage of MCC-257 used to treat a particular condition may vary in practice due to a wide variety of factors.

Accordingly, dosage guidelines provided herein are not limiting as the range of actual dosages, but rather provide guidance to skilled practitioners in selecting dosages useful in the empirical determination of dosages for individual patients. Advantageously, methods described herein allow treatment of one or more conditions with reductions in side effects, dosage levels, dosage frequency, treatment duration, safety, tolerability, and/or other factors. Suitable dosages for MCC-257 for other indications are known to a skilled person. The disclosure includes the use of about 75%, about 50%, about 33%, about 25%, about 20%, about 15%, about 10%, about 5%, about 2.5%, about 1%, about 0.5%, about 0.25%, about 0.2%, about 0.1%, about 0.05%, about 0.025%, about 0.02%, about 0.01%, or less than the known dosage.

In other embodiments, the amount of MCC-257 used in vivo may be about 50%, about 45%, about 40%, about 35%, about 30%, about 25%, about 20%, about 18%, about 16%, about 14%, about 12%, about 10%, about 8%, about 6%, about 4%, about 2%, or about 1% or less than the maximum tolerated dose for a subject.

Alternatively, the amount of MCC-257 may be an amount selected to be effective to produce an improvement in a treated subject based on detectable neurogenesis in vitro as described above. With MCC-257 the amount is one that minimizes clinical side effects seen with administration of the agent to a subject. The amount of an agent used in vivo may be about 50%, about 45%, about 40%, about 35%, about 30%, about 25%, about 20%, about 18%, about 16%, about 14%, about 12%, about 10%, about 8%, about 6%, about 4%, about 2%, or about 1% or less of the maximum tolerated dose in terms of acceptable side effects for a subject. This is readily determined for MCC-257 through clinical use or testing, such as in humans.

In other embodiments, the amount of MCC-257 may be the highest amount which produces no detectable neurogenesis in vitro, including in vivo animal (or non-human) models for behavior linked to neurogenesis, but yet produces neurogenesis, or a measurable shift in efficacy in promoting neurogenesis in the in vitro assay. Embodiments include amounts which produce about 1%, about 2%, about 4%, about 6%, about 8%, about 10%, about 12%, about 14%, about 16%, about 18%, about 20%, about 25%, about 30%, about 35%, or about 40% or more of the neurogenesis seen with the amount of MCC-257 that produces the highest level of neurogenesis in an in vitro assay.

In some embodiments, the amount may be the lowest needed to produce a desired, or minimum level of detectable neurogenesis or beneficial effect. The MCC-257 administered, may be in the form of a pharmaceutical composition.

As described herein, the amount of MCC-257, may be any that is effective to produce neurogenesis.

In some embodiments, an effective, neurogenesis modulating amount of MCC-257 is an amount that achieves a concentration within the target tissue, using the particular mode of administration, at or above the IC₅₀ or EC₅₀ for activity of target molecule or physiological process. In some cases, MCC-257 may be administered in a manner and dosage that gives a peak concentration of about 1, about 1.5, about 2, about 2.5, about 5, about 10, about 20 or more times the IC₅₀ or EC₅₀ concentration of MCC-257. IC₅₀ and EC₅₀ values and bioavailability data for MCC-257, are known in the art and are described, e.g., in the references cited herein or may be readily determined using established methods. In addition, methods for determining the concentration of a free compound in plasma and extracellular fluids in the CNS, as well pharmacokinetic properties, are known in the art, and are described, e.g., in de Lange et al., AAPS Journal, 7(3): 532-543 (2005). In some embodiments, MCC-257 may be administered at a frequency of at least once a week, about once daily, or about twice daily, or about three or more times daily, and for a duration of at least about 3 days, about 5 days, about 7 days, about 10 days, about 14 days, or about 21 days, or about 4 weeks, or about 2 months, or about 4 months, or about 6 months, or about 8 months, or about 10 months, or about 1 year, or about 2 years, or about 4 years, or about 6 years or longer.

In other embodiments, an effective, neurogenesis modulating amount of MCC-257, is a dose that produces a concentration in an organ, tissue, cell, and/or other region of interest that includes the ED₅₀ (the pharmacologically effective dose in 50% of subjects) with little or no toxicity. IC₅₀ and EC₅₀ values for the modulation of neurogenesis may be determined using methods described in U.S. patent publication 2007/0015138 to Barlow et al., filed Jul. 8, 2005, incorporated by reference, or by other methods known in the art. In some embodiments, the IC₅₀ or EC₅₀ concentration for the modulation of neurogenesis is substantially lower than the IC₅₀ or EC₅₀ concentration for activity of MCC-257 at non-targeted molecules and/or physiological processes.

In some methods, the application of MCC-257, may allow effective treatment with substantially fewer and/or less severe side effects compared to existing treatments. In further embodiments, the disclosed methods allow treatment of certain conditions for which treatment with the same or similar compounds is ineffective, for example, to dose-limiting side effects, toxicity, and/or other factors.

In some embodiments, the methods of treatment disclosed herein comprise the step of administering MCC-257, to a mammal or more preferably man, for a time and at a concentration sufficient to treat the condition targeted for treatment. The disclosed methods may be applied to individuals having, or who are likely to develop, disorders relating to neural degeneration, neural damage and/or neural demyelination.

Depending on the desired clinical result, the disclosed agents or pharmaceutical compositions may be administered by any means suitable for achieving a desired effect. Various delivery methods are known in the art and may be used to deliver the agent or composition to a subject or to NSCs or progenitor cells within a tissue of interest. The delivery method will depend on factors such as the tissue of interest, the nature of the compound (e.g., its stability and ability to cross the blood-brain barrier), and the duration of the experiment or treatment, among other factors. For example, an osmotic mini-pump may be implanted into a neurogenic region, such as the lateral ventricle. Alternatively, compounds may be administered by direct injection into the cerebrospinal fluid of the brain or spinal column, or into the eye. Compounds can also be administered into the periphery (such as by intravenous or subcutaneous injection, or oral delivery), and subsequently cross the blood-brain barrier.

In some embodiments, the disclosed agents or pharmaceutical compositions are administered in a manner that allows them to contact the subventricular zone (SVZ) of the lateral ventricles and/or the dentate gyrus of the hippocampus. The delivery or targeting of MCC-257, to a neurogenic region, such as the dentate gyrus or the subventricular zone, may enhance efficacy and reduce side effects compared to known methods involving administration with the same or similar compounds. Examples of routes of administration include parenteral, e.g., intravenous, intradermal, subcutaneous, oral (e.g., inhalation), transdermal (topical), transmucosal, and rectal administration. Intranasal administration generally includes, but is not limited to, inhalation of aerosol suspensions for delivery of compositions to the nasal mucosa, tracheand bronchioli.

In other embodiments, MCC-257, may be administered so as to either pass through or by-pass the blood-brain barrier. Methods for allowing factors to pass through the blood-brain barrier are known in the art, and include minimizing the size of the factor, providing hydrophobic factors which facilitate passage, and conjugation to a carrier molecule that has substantial permeability across the blood brain barrier. In some instances, MCC-257, may be administered by a surgical procedure by implanting a catheter coupled to a pump device. The pump device can also be implanted or be extracorporally positioned. Administration of MCC-257, may be in intermittent pulses or as a continuous infusion. Devices for injection to discrete areas of the brain are known in the art. In certain embodiments, MCC-257, may be administered locally to the ventricle of the brain, substantia nigra, striatum, locus ceruleous, nucleus basalis Meynert, pedunculopontine nucleus, cerebral cortex, and/or spinal cord by, e.g., injection. Methods, compositions, and devices for delivering therapeutics, including therapeutics for the treatment of diseases and conditions of the CNS and PNS, are known in the art.

In some embodiments, MCC-257 may be modified to facilitate crossing of the gut epithelium. For example, in some embodiments, MCC-257 in a prodrug form is transported across the intestinal epithelium and metabolized into the active agent in systemic circulation and/or in the CNS.

In other embodiments, MCC-257 may be conjugated to a targeting domain to form a chimeric therapeutic, where the targeting domain facilitates passage of the blood-brain barrier (as described above) and/or binds one or more molecular targets in the CNS. In some embodiments, the targeting domain binds a target that is differentially expressed or displayed on, or in close proximity to, tissues, organs, and/or cells of interest. In some cases, the target is distributed in a neurogenic region of the brain, such as the dentate gyrus and/or the SVZ. For example, in some embodiments, MCC-257 may be conjugated or complexed with the fatty acid docosahexaenoic acid (DHA), which is readily transported across the blood brain barrier and imported into cells of the CNS.

Having now generally described the disclosure, the same will be more readily understood through reference to the following examples which are provided by way of illustration, and are not intended to be limiting to the disclosure, unless otherwise specified.

### EXAMPLES

### Example 1

### Effects of MCC-257 on Neuronal Differentiation of Human Neural Stem Cells

Human neural stem cells (hNSCs) were isolated and grown in monolayer culture, plated, treated with varying concentrations of MCC-257, and stained with TUJ-1 antibody for the detection of neuronal differentiation as described in U.S. Patent Publication 2007/0015138 (incorporated by reference). Mitogen-free test media with a positive control for neuronal differentiation was used along with basal media without growth factors as a negative control.

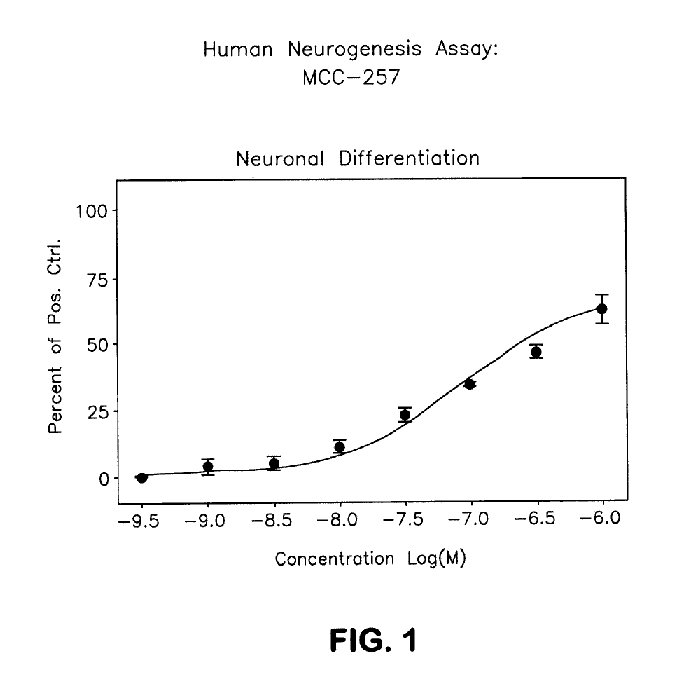

Results are shown in FIG. 1, which shows a concentration response curve of neuronal differentiation after background media values are subtracted. The data is presented as a percent of neuronal positive control. The data indicate that MCC-257 promoted neuronal differentiation in a concentration dependent manner.

### Example 2

### In Vivo Chronic Dosing Studies of MCC-257

Novelty-Suppressed Feeding Test (NSF)

Few behavioral paradigms have been able to reliably demonstrate changes in rodent behavior after chronic, but not acute, treatment with antidepressant drugs (Cryan et al. “Assessing antidepressant activity in rodents: recent developments and future needs.” 2002Trends Pharmacol Sci 23(5):238-45). The NSF is a conflict test in which food-deprived animals are presented with a food pellet placed in the center of a brightly-lit open field. This paradigm elicits competing motivations: the drive to eat the food pellet, and the fear of venturing into the center of the arena. The dependent variable in this test is the amount of time it takes for an animal to begin eating the pellet, and this latency has been used as an index of anxiety-like behavior and antidepressant activity because it is decreased by classical anxiolytic drugs such as benzodiazepines (Bodnoff et al. “The effects of chronic antidepressant treatment in an animal model of anxiety.” 1988 Psychopharmacology (Berl) 95(3):298-302; Bodnoff et al. “A comparison of the effects of diazepam versus several typical and atypical anti-depressant drugs in an animal model of anxiety.” 1989 Psychopharmacology (Berl) 97(2):277-9; Shephard and Broadhurst, “Hyponeophagia and arousal in rats: effects of diazepam, 5-methoxy-N,N-dimethyltryptamine, d-amphetamine and food deprivation.” 1982 Psychopharmacology (Berl) 78(4):386-72) as well as antidepressants.

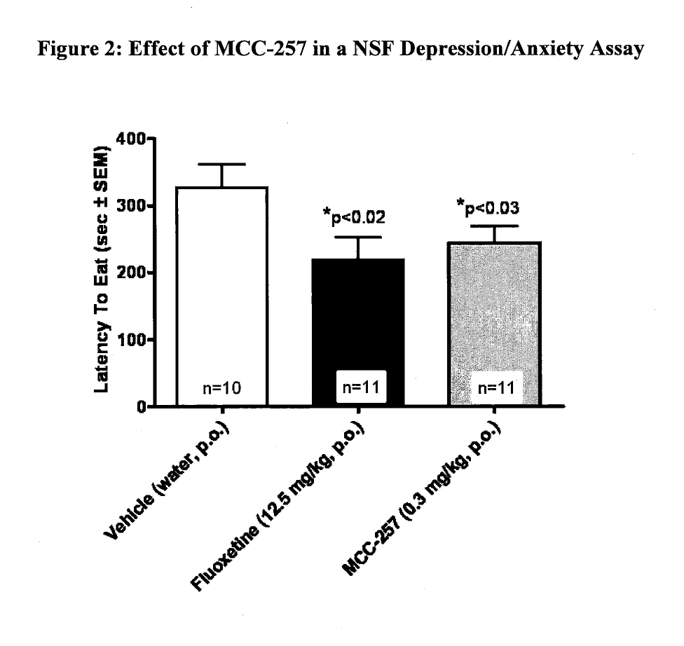

NSF is performed as described previously by Santarelli, et al. (2003) with some modifications. Briefly, male F344 rats were dosed orally once daily (q.d.) for 20-days with vehicle (DI water; n=10), 12.5 mg/kg fluoxetine (n=11) or 0.3 mg/kg MCC-257 (n=11). Animals were food deprived for 24 hours prior to testing. On day 22, 60 minutes after drug administration, NSF was performed in an open field chamber. A single pellet of rodent chow was placed in the center of the open field. Animals were placed in a corner of the open field and video recording was initiated immediately. Saved videos were subsequently scored by blinded observers and the latency to eat the pellet was recorded. FIG. 2 shows the mean latency to approach and eat a food pellet within the novel environment. Both fluoxetine (12.5 mg/kg) and MCC-257 (0.3 mg/kg) significantly reduced (p<0.05, one-tailed Student's t-test) the latency to eat the food pellet indicating antidepressant/anxiolytic activity of both compounds.

### In Vivo Neurogenesis

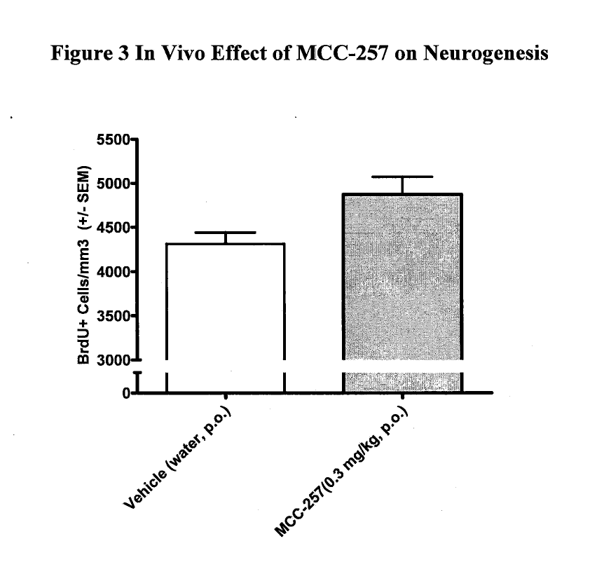

For the in vivo neurogenesis assays, male F344 rats were dosed orally once daily (q.d.) for 28-days with vehicle (DI water; n=11) or 0.3 mg/kg MCC-257 (n=12). BrdU was administered once daily between days 9 and 14 (100 mg/kg/day, i.p.). FIG. 3 shows BrdU positive cell counts per cubic mm within the granule cell layer of the dentate gyrus. Chronic dosing of MCC-257 (0.3 mg/kg) resulted in a significant increase (p<0.5, one-tailed Student's t-test) in BrdU positive cells compared to vehicle.

Novel Object Recognition Assay (NOR)

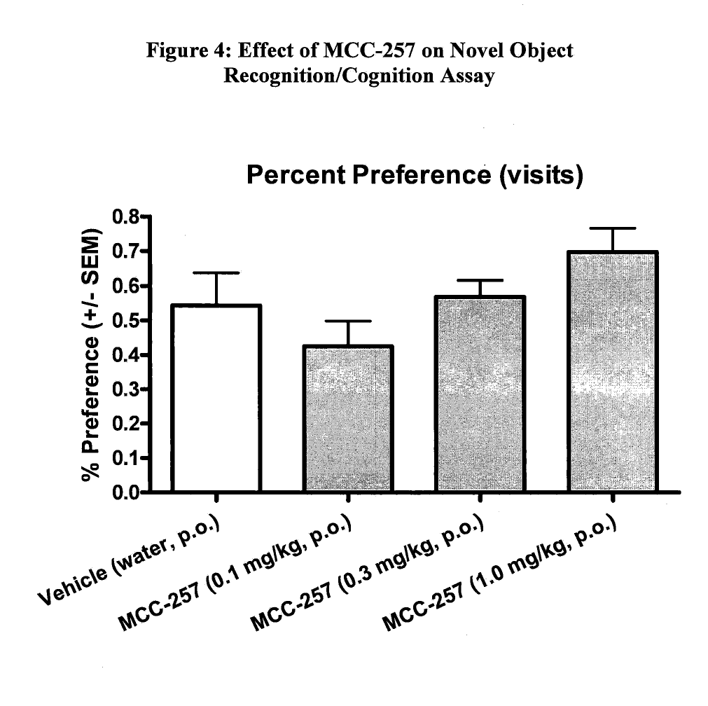

NOR was performed as described previously by Broadbent et al., (“Spatial memory, recognition memory, and the hippocampus.” 2004 PNAS 101(40):14511-14520) with some modifications. The apparatus consisted of an open field (45×45×50 cm high) made of polycarbonate. Triplicate copies were used of the objects to be discriminated. Care was taken to ensure that the pair of objects tested were made from the same material so that they could not be distinguished readily by olfactory cues although they had very different appearances. Each test session consisted of two phases. In the initial familiarization phase, two identical objects (A1 and A2) were placed in the far corners of the box arena. A rat was then placed in the middle of the arena and allowed 15 minutes to explore both objects. Exploration of an object was defined as directing the nose to the object at a distance of less than 2 cm and/or touching it with the nose. After a delay of 48-hours the rat was re-introduced to the arena (“test phase”). The box now contained a third identical copy of the familiar object (A3) and a new object (B). These were placed in the same locations as the sample stimuli, whereby the position (left or right) of the novel object in the test phase was balanced between rats. For half the rats, object A was the sample and object B was the novel alternative. The test phase was 15 minutes in duration, with the first 30 seconds of object interaction used to determine preference scores. Any animal with less than 15 seconds of object exploration were excluded from analysis. The percent time of exploring the novel object and/or percent visits to the novel object serve as a measure of recognition memory for the familiar object. Male F344 rats were dosed orally once daily (q.d.) for 26-days with vehicle (DI water, n=10) or 0.1, 0.3 and 1.0 mg/kg MCC-257 (n=11). FIG. 4 shows the mean percent preference based on visits to the novel object. MCC-257 produced a dose dependent increase in preference for the novel object indicative of cognitive enhancement with chronic MCC-257 treatment.

All references cited herein, including patents, patent applications, and publications, are hereby incorporated by reference in their entireties, whether previously specifically incorporated or not.

Having now fully provided the instant disclosure, it will be appreciated by those skilled in the art that the same may be performed within a wide range of equivalent parameters, concentrations, and conditions without departing from the spirit and scope of the disclosure and without undue experimentation. While the disclosure has been described in connection with specific embodiments thereof, it will be understood that it is capable of further modifications. This application is intended to cover any variation, use, or adaptation of the disclosure following, in general, the disclosed principles and including such departures from the disclosure as come within known or customary practice within the art to which the disclosure pertains and as may be applied to the essential features hereinbefore set forth.

What is claimed is:

1. A method of treating a subject suffering from a nervous system disorder comprising administering to the subject a therapeutically effective amount of 5-acetamido-N-(5alpha-cholestan-3alpha-yl)-3,5-dideoxy-2-O-methyl-D-glycero-alpha-D-galacto-non-2-ulopyranosonamide, or a pharmaceutically-acceptable hydrate or solvate thereof.

2. A method of claim 1, wherein the subject is an animal or human.

3. A method of claim 1, wherein the nervous system disorder is a mental disorder or a disease of the central nervous system.

4. A method of claim 3, wherein the mental disorder is an affective disorder.

5. A method of claim 3, wherein the disease of the central nervous system causes cognitive impairment.

6. A method of claim 5 wherein cognitive impairment is the result of chronic infection, toxic disorders, neurodegenerative disorders, or combinations thereof.

7. The method of claim 1, wherein 5-acetamido-N-(5alpha-cholestan-3alpha-yl)-3,5-dideoxy-2-O-methyl-D-glycero-alpha-D-galacto-non-2-ulopyranosonamide is in a pharmaceutically acceptable formulation.

8. A method of stimulating or increasing neurogenesis in a cell or tissue, the method comprising contacting the cell or tissue with an effective amount of 5-acetamido-N-(5alpha-cholestan-3alpha-yl)-3,5-dideoxy-2-O-methyl-D-glycero-alpha-D-galacto-non-2-ulopyranosonamide, or a pharmaceutically acceptable hydrate or solvate thereof, to stimulate or increase neurogenesis in said cell or tissue.

9. The method of claim 8, wherein the cell or tissue is in an animal or a human subject.

10. The method of claim 9, wherein the subject has a condition, affecting normal neurogenesis whereby stimulating or increasing neurogenesis improves the condition.

11. The method of claim 10, wherein the condition causes decreased neurogenesis in the subject.

12. The method of claim 10, wherein the condition causes aberrant neurogenesis in the subject.

13. The method of claim 10, wherein the subject had been exposed to an agent causing decreased neurogenesis.

14. The method of claim 8, wherein the neurogenesis comprises differentiation of neural stem cells (NSCs) along a neuronal lineage.

15. The method of claim 8, wherein the neurogenesis comprises differentiation of neural stem cells (NSCs) along a glial lineage.

16. The method of claim 9, wherein the neurogenesis occurs in the hippocampus of the subject.

17. The method of claim 8, wherein 5-acetamido-N-(5alpha-cholestan-3alpha-yl)-3,5-dideoxy-2-O-methyl-D-glycero-alpha-D-galacto-non-2-ulopyranosonamide is in a pharmaceutically acceptable formulation.

18. A method of treating a subject suffering from cognitive impairment due to a non-disease state comprising administering to the subject a therapeutically-effective amount of 5-acetamido-N-(5alpha-cholestan-3alpha-yl)-3,5-dideoxy-2-O-methyl-D-glycero-alpha-D-galacto-non-2-ulopyranosonamide, or a pharmaceutically-acceptable hydrate or solvate thereof.

19. The method of claim 18, wherein the non-disease state is aging.

20. The method of claim 18, wherein the cognitive impairment is a result of chemotherapy or radiation therapy.
